(19) United States
(12) Patent Application Publication
(10) Pub. No.: US 2002/0072051 A1
(43) Pub. Date: Jun. 13, 2002

(54) Screen for identifying inhibitors of GPI anchoring

(76) Inventors: Christine Ellen Bulawa, Arlington, MA (US); Marie Keaveney, Salem, MA (US); Ronald K. Blackman, Brookline, MA (US)

Correspondence Address: J. PETER FASSE, FISH & RICHARDSON P.C., 225 Franklin Street, Boston, MA, 02110-2804, US

(21) Appl. No.: 09/887,489

(22) Filed: Jun. 22, 2001

Related U.S. Application Data
(60) Provisional application No. 60/213,623, filed on Jun. 23, 2000.

(51) Int. Cl. C12Q 1/00; C12Q 1/66; C12Q 1/02; C12N 1/18

(52) U.S. Cl. 435/4; 435/8; 435/29; 435/254.21

(57) ABSTRACT

Methods for identifying compounds that are capable of activating the UPR pathway, inhibition of glycosylphosphatidylinositol (GPI) anchoring, and/or antifungal activity are disclosed. Also disclosed are methods for treating fungal infections in an organism using compounds identified as having antifungal activity, and methods for treating a protozoan infection in an organism using compounds identified as inhibiting GPI anchoring.

### CROSS REFERENCE TO RELATED APPLICATION

This application claims priority from U.S. Provisional patent application Ser. No. 60/213,623, filed on Jun. 23, 2000, which is incorporated herein by reference in its entirety.

### TECHNICAL FIELD

This invention relates to molecular biology, cell biology, mycology and drug discovery.

### BACKGROUND

The Unfolded Protein Response pathway (UPR pathway) is activated by the accumulation of unfolded proteins in the lumen of the endoplasmic reticulum (ER). Induced proteins in the UPR pathway include molecular chaperones and protein folding enzymes localized in the ER. Activation of the UPR pathway is triggered by N-linked glycosylation inhibitors (tunicamycin), reducing agents (dithiothreitol), and the expression of mutant secretory proteins that are unable to fold correctly.

Tunicamycin is an efficient inhibitor of N-linked glycosylation and is a fungicidal compound. The resulting non-glycosylated proteins are not efficiently processed in the endoplasmic reticulum. The increased ratio of unfolded proteins-to-endoplasmic reticulum membrane triggers UPR pathway activation. Several genes are induced in the unfolded protein response pathway. Genes designated KAR2, LHS1, and PDI1 have been identified in the UPR pathway in Saccharomyces cerevisiae. Each of these genes contains a common upstream promoter element known as the UPR element (UPRE).

Many cell surface proteins are anchored to the cell membrane by a glycosylphosphatidylinositol (GPI) moiety, which is attached to the C terminus of the proteins. GPI anchor biosynthesis is a complicated 12-step process in which a cell progressively decorates phosphatidylinositol (PI) with various sugar residues to generate the complete precursor, which can then be covalently attached to a protein. The core of the GPI anchor appears to be highly conserved among eukaryotes, but it has variable side chains (Hong et al., 1999, J. Biol. Chem. 274:35099-35106). In yeast, GPI-anchored proteins are found on the plasma membrane, but also as an intrinsic part of the cell wall. Although no single GPI-anchored protein is essential, loss of all GPI-anchored proteins by blocking GPI-anchor precursor synthesis is lethal. The synthesis of GPI-anchors is conserved through evolution; however, there are still differences between fungal and mammalian anchor synthesis that provide an avenue for selectivity. For example, the MCD4 catalyzed step is essential in yeast but is dispensable in mammalian cells. A known inhibitor of GPI anchor biosynthesis is a terpenoid lactone designated CJ-19089 (also known as YW3548) (Sutterlin et al., 1997, EMBO J. 16:6374-6383).

### SUMMARY

There remains a need for antifungal agents that are active against fungi but are minimally toxic to mammalian cells. Accordingly, there is a need for an assay or screening method that specifically identifies those agents that are active against specific intracellular targets in fungi. The present invention provides screening methods for identifying compounds that are capable of one or more of the following activities in fungi: the ability to activate the UPR pathway, inhibition of glycosylphosphatidylinositol (GPI) anchoring, and antifungal activity (fungistatic and/or fungicidal activity).

In one aspect, the invention provides a method for identifying compounds that activate the unfolded protein response pathway in fungi. The method for identifying a compound that activates the UPR pathway includes: (a) providing a yeast cell containing a vector comprising at least one unfolded protein response element operably linked to a reporter element; (b) incubating the yeast cell in the presence of a candidate compound; and (c) detecting expression of the reporter element in the presence of the candidate compound as compared to expression of the reporter element in the absence of the candidate compound. A 2-fold or greater increase in expression of the reporter element in the presence of the candidate compound indicates that the candidate compound activates the unfolded protein response pathway.

In one embodiment, the method for identifying a compound that activates the unfolded protein response pathway further includes a secondary screen. The secondary screen includes subjecting the compound that activates the unfolded protein response pathway to an assay for the inhibition of GPI anchoring. In another embodiment, the secondary screen comprises an enzyme assay for a step in GPI anchor biosynthesis or an assay for maturation for a yeast GPI-anchored protein. The secondary screen can comprise an assay for detecting inositol incorporation into protein by yeast cells. The secondary screen can also be an overexpression resistance assay. In yet another embodiment, the secondary screen can be a lipid labeling assay. The lipid labeling assay can include labeling lipids with [³H]-inositol, [¹⁴C]-mannose, or both.

In other embodiments, the yeast cell can be a member of the genus Candida or Saccharomyces. The UPRE can include a nucleotide sequence such as: AGGAACTGGACAGCGTGTCGAAA (SEQ ID NO: 1). The vector can contain one to five UPREs, e.g., 3 UPREs, operably linked to a reporter element. The skilled practitioner will appreciate that the reporter element can be any reporter element known in the art. Exemplary reporter elements are a β-galactosidase coding sequence, a luciferase coding sequence, or a green fluorescent protein coding sequence.

In another aspect, the invention provides a method for identifying compounds having antifungal activity. The method for identifying such compounds includes: (a) providing a yeast cell containing a vector comprising at least one unfolded protein response element operably linked to a reporter element; (b) incubating the yeast cell in the presence of a candidate compound; (c) detecting expression of the reporter element in the presence of the candidate compound as compared to expression of the reporter element in the absence of the candidate compound, wherein a 2-fold or greater increase in expression of the reporter element in the presence of the candidate compound indicates that the candidate compound activates the unfolded protein response pathway; and (d) assaying a compound that activates the unfolded protein response pathway to an assay for inhibition of GPI anchoring. The inhibition of GPI anchoring in the GPI anchoring assay indicates that the compound has antifungal activity.

In certain embodiments, the method further includes testing the compound directly for antifungal activity. The compound can be tested for antifungal activity using any method known in the art involving exposing the fungus to the compound and observing the effect of the compound on fungal growth. For example, the compound is tested for antifungal activity using a halo assay. The halo assay can be performed at any given step of the method.

In another embodiment, the GPI anchoring assay comprises an enzyme assay for a step in GPI anchor biosynthesis or an assay for maturation for a yeast GPI-anchored protein. The GPI anchoring assay can comprise an assay for detecting inositol incorporation into protein by yeast cells. The GPI anchoring assay can also be an overexpression resistance assay. In yet another embodiment, the GPI anchoring assay can be a lipid labeling assay. The lipid labeling assay can include labeling lipids with [³H]-inositol, [¹⁴C]-mannose, or both.

In other embodiments, the yeast cell can be a member of the genus Candida or Saccharomyces. The UPRE can include a nucleotide sequence such as: AGGAACTGGACAGCGTGTCGAAA (SEQ ID NO: 1). Preferably, the vector contains one to five UPREs, e.g., 3 UPREs, operably linked to a reporter element. The skilled practitioner will appreciate that the reporter element can be any reporter element known in the art. Exemplary reporter elements are a β-galactosidase coding sequence, a luciferase coding sequence, or a green fluorescent protein coding sequence.

In yet another aspect, the invention provides a method for treating fungal infections in an organism. The method includes administering to the organism a therapeutically effective amount of a compound identified as having antifungal activity by a method of the present invention. In certain embodiments, the fungal infection can be fungal dermatophytoses, pulmonary disorders caused by hypersensitivity to fungi, fungal infections with pleural involvement, fungal infections involving the genitourinary tract, and/or systemic mycoses.

In another aspect, the invention provides a method for treating a protozoan infection in an organism. The method includes administering to the organism a therapeutically effective amount of a compound identified as inhibiting GPI anchoring by a method of the present invention. For example, the protozoan infection can be amebiasis, giardiasis, malaria, leishmaniasis, babeosiosis, and/or cryptosporidiosis

Unless otherwise defined, all technical and scientific terms used herein have the same meaning as commonly understood by one of ordinary skill in the art to which this invention belongs. In case of conflict, the present application, including definitions, will control. All publications, patents and other references mentioned herein are incorporated by reference.

Although methods and materials similar or equivalent to those described herein can be used in the practice or testing of the present invention, the preferred methods and materials are described below. The materials, methods and examples are illustrative only and not intended to be limiting. Other features and advantages of the invention will be apparent from the detailed description and from the claims.

### DESCRIPTION OF DRAWINGS

FIG. 1 is a representation of the nucleic acid sequence of an Unfolded Protein Response Pathway Element (UPRE; SEQ ID NO:1)

FIG. 2 is a representation of the nucleic acid sequence of the 3X UPRE-CYCl minimal promoter cloned into a pRS416 vector (SEQ ID NO:2).

### DETAILED DESCRIPTION

In the present invention, a novel combination of known elements or steps has been assembled into efficient screening methods for identifying compounds that inhibit GPI anchor biosynthesis. Practice of the new methods generally involves two stages: first, an induction screen, which indicates whether or not a test or candidate compound induces the UPR pathway, and second, a biochemical assay, which indicates whether or not a candidate that induces the UPR pathway also inhibits GPI anchoring.

The novel screening methods represent an advantageous approach for rapidly discovering anti-fungal drug candidates. The new methods reflect a surprising discovery in that compounds that kill or inhibit the growth of yeast can be identified at levels below their respective minimum inhibitory concentrations (MIC) by assaying for the induction of UPRE-regulated gene expression. Previous screens for fungistatic and fungicidal compounds were based on detecting inhibition; the present methods are based on detecting induction.

The new methods feature various advantages. For example, the methods are used to detect induction of UPRE-regulated gene expression, rather than initially observing inhibition of GPI anchoring by assaying for activity of an enzyme used for GPI anchor biosynthesis, maturation of GPI-anchored proteins, or some other assay specific for GPI anchoring.

Other advantages include efficiency (e.g., single screen detects inhibitors acting at any of various points in pathway), ease of use, good quantitation, sensitivity (e.g., effective drugs can be detected at concentrations below the MIC), reliability, reproducibility, selectivity, facility, versatility (e.g., the methods are adaptable from benchtop to high throughput screening methodology), and robustness (e.g., the screening methods can use natural product extracts which are impure).

The high sensitivity of the new methods provides for the discovery of compounds that are neither fungistatic nor fungicidal but nonetheless affect growth of yeast. Although such compounds might not themselves be effective drugs, they can be used to lead to novel drugs. For example, the compounds discovered by any of the new methods can serve as a basis for the design of structural analogs, some of which are likely to be more effective than the initially discovered compounds. The structural analogs can also be screened by the new methods.

Furthermore, the new methods allow screening for inhibitors of reactions, in addition to inhibitors of enzymes. This is important for at least three reasons. First, some potential antifungal compounds bind to the substrate of a reaction thereby rendering that substrate unavailable for reaction with an enzyme. The enzyme itself is not affected by the inhibitor; nonetheless the observed result is the same (i.e., the enzymatic reaction is ceased). Second, multiple steps in GPI anchoring can be carried out by a single, multiple domain enzyme, while certain inhibitors can block the activity of just one of the domains. Third, some proteins in the pathway are not enzymes.

The methods of the invention are suitable for high throughput screening formats and very high throughput screening formats. The new methods can be carried out in nearly any reaction vessel or receptacle. Examples of suitable receptacles include 96-well plates, 384-well plates, test tubes, centrifuge tubes, and microcentrifuge tubes. The methods can also be carried out on surfaces such as on metal, glass, or polymeric chips, membrane surfaces, the surface of a matrix-assisted laser-desorption ionization mass spectrometry (MALDI-MS) plate, on a resin, and on a glass, metal, ceramic, paper, or polymer surface.

Guidance concerning various components of the methods, i.e., yeast cells, induction screen, UPREs, reporter elements, vectors and secondary screens, are discussed, in turn, in the paragraphs below.

Induction Screen

The first stage of the new antifungal screening methods uses UPRE directed induction of the reporter as a signal of antifungal activity. The screen can be carried out in yeast that carry a UPRE operably linked to a reporter sequence.

The yeast are contacted (e.g., incubated) with a candidate compound. The candidate compound can be, for example, a single compound or a member of a library of potential inhibitors. In some embodiments of the invention, the candidate compound is a compound not previously known to inhibit fungal growth. In other embodiments, the candidate compound is a known antifungal compound, and the invention is employed to obtain information on the known compound's mode of action, e.g., whether the mode of action involves GPI anchoring, and if so, what step in GPI-anchor biosynthesis or the GPI-anchoring process is being targeted.

Incubation times vary with yeast species (or strain) and incubation temperature (e.g., 1 hour, 12 hours, 1 day, 2 days, a week, or longer). Suitable conditions that normally allow UPRE induction can include aerobic or anaerobic atmospheres at room temperature or lower, 30° C., 37° C., or higher, depending on the species of fungi.

A library of potential inhibitors can be a synthetic combinatorial library (e.g., a combinatorial chemical library), a cellular extract, a bodily fluid (e.g., urine, blood, tears, sweat, or saliva), or other mixture of synthetic or natural products (e.g., a library of small molecules or a fermentation mixture).

A library of potential inhibitors can include, for example, amino acids, oligopeptides, polypeptides, proteins, or fragments of peptides or proteins; nucleic acids (e.g., antisense; DNA; RNA; or peptide nucleic acids, PNA); aptamers; or carbohydrates or polysaccharides. Each member of the library can be singular or can be a part of a mixture (e.g., a compressed library), or organic or inorganic small molecules The library can contain purified compounds or can be “dirty” (i.e., containing a significant quantity of impurities).

Commercially available libraries (e.g., from Affymetrix, ArQule, Neose Technologies, Sarco, Ciddco, Oxford Asymmetry, Maybridge, Aldrich, Panlabs, Pharmacopoeia, Sigma, or Tripose) can also be used with the new methods.

In addition to libraries of potential inhibitors, special libraries called diversity files can be used to assess the specificity, reliability, or reproducibility of the new methods. Diversity files contain a large number of compounds (e.g., 1000 or more small molecules) representative of many classes of compounds that could potentially result in nonspecific detection in an assay. Diversity files are commercially available or can also be assembled from individual compounds commercially available from the vendors listed above.

Assays are then carried out to determine the level of UPRE induction and thus the effectiveness of the inhibitors. In general, the higher the level of induction, the higher the level of effectiveness of a given inhibitor candidate. Assays for UPRE induction through the detection of a reporter gene product can be carried out, for example, using fluorimetry, spectrophotometry (e.g., by measuring the optical absorbance of the reaction mixture), measurement of light emitted by a bioluminescence enzyme (e.g., using a luminometer), antibodies that specifically bind to a polypeptide encoded by a UPRE-linked reporter sequence, or by probing for reporter mRNA (e.g., using a labeled probe; the label can be, for instance, fluorescent, radioactive, or biotinylated). Spectroscopic methods (e.g., high performance liquid chromatography, HPLC) can also be used, as can electrophoresis (agarose gel, polyacrylamide gel electrophoresis, etc.) or affinity chromatography. In another alternative, labeled substrates can be used to assay for UPRE-driven reporter expression.

Yeast Cells

Various species of fungi can be employed as host cells in a screening method according to the invention. Useful species include, for example, Microsporum canis, Trichophyton rubrum, Trichophyton mentagrophytes, Candida albicans, Candida tropicalis, Saccharomyces cerevisiae, Torulopsis glabrata, Pichia pastoris, Epidermophyton floccosum, Malasseziafurfur, Pityropsporon orbiculare, Pityropsporon ovale, Cryptococcus neoformans, Aspergillus fumigatus, Aspergillus nidulans, Paracoccidioides brasiliensis, Blastomyces dermatitides, Histoplasma capsulatum, Coccidioides immitis, and Sporothrix schenckii. In some embodiments of the invention, a Δmdr yeast strain is employed. Suitable yeast strains are commercially available. For example, they can be obtained from the American Type Culture Collection (ATCC), Rockville, Md. Methods and materials for laboratory culture of yeast cells is well known in the art. A preferred yeast strain is one with the following genotype: MATa/(α; ura3Δ0/ura3Δ0; leu2Δ0/leu2Δ0; his3Δ1/his3Δ1; met15Δ0/MET15; LYS2/lys2Δ0; pdr5Δ::HIS3/pdr5Δ::HIS3; snq2Δ::HIS3/snq2Δ::HIS3.

UPRE

A basic component of the present invention is a UPRE. For information concerning UPREs in general, see, e.g., Shamu et al., 1994, Trends in Cell Biol. 4:56-60. There is no requirement for a particular UPRE, i.e., a specific nucleotide sequence. Preferably, the UPRE is a yeast UPRE. Typically, a yeast UPRE consists of about 20-25 nucleotides. A specific example of a yeast UPRE suitable for use in the present invention is the following 23-nucleotide sequence: AGGAACTGGACAGCGTGTCGAAA (SEQ ID NO:1). As taught in PCT publication WO 96/08561, another specific example of a yeast UPRE is nucleotides 2-23 of SEQ ID NO: 1. A UPRE useful in the invention can be obtained by any suitable means. The UPRE can be chemically synthesized, using conventional methods and materials. For example, the UPRE can be synthesized by employing an automated, commercial DNA synthesizer according to the vendor's recommendations.

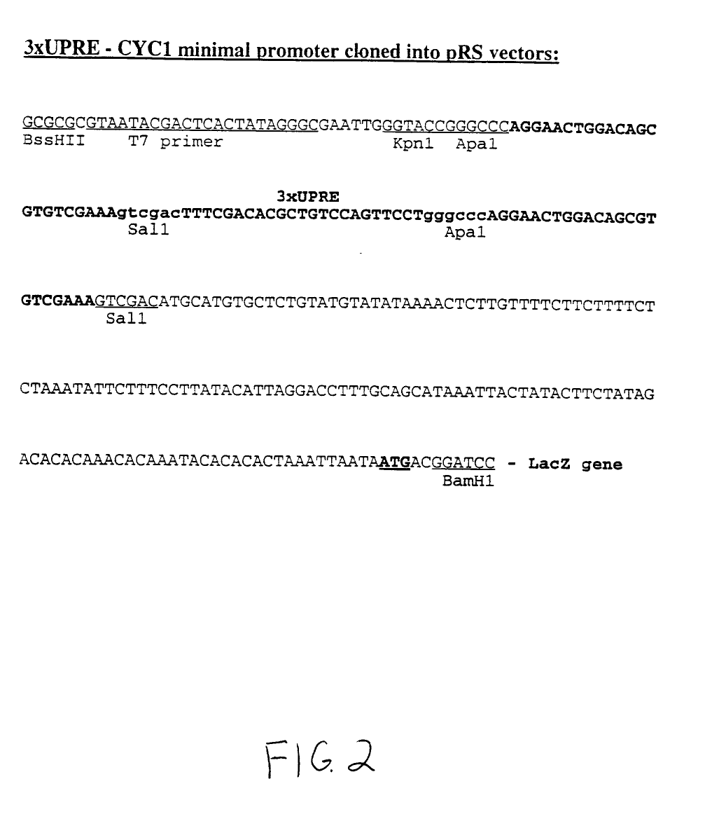

Those of skill in the art will appreciate that the UPRE can be synthesized with pre-selected flanking sequences. For example, in some embodiments, 2-6 nucleotides are added to the 5′ end of one strand of the UPRE, and 2-6 nucleotides are added to the 3′ end of the complementary strand, so that hybridization of the two strands results in a short overhang at each end of the double-stranded UPRE, i.e., “sticky ends.” This facilitates insertion of the UPRE into a restriction site during vector construction. In a preferred embodiment of the invention, the UPRE is flanked by an Apal site at its 5′ end, and a SaII site at its 3′ end (FIG. 2). In another example, a UPRE is flanked at both ends by XhoI sites (WO 96/08561).

Reporter Element

A reporter element or gene is a nucleotide sequence encoding a detectable polypeptide. In some embodiments, the reporter gene does not occur naturally in the yeast strain employed in the screen. Preferably the reporter gene encodes a polypeptide detectable by established methods. Preferably, the polypeptide encoded by the reporter gene provides a readout compatible with an automated high throughput screening system. Suitable readouts include a calorimetric reaction, luminescence, and fluorescence. Exemplary reporter elements include lacZ, which encodes β-galactosidase (a colorimetric enzyme); luc, which encodes luciferase (a bioluminescent enzyme); and a GFP gene, which encodes green fluorescent protein (a fluorescent protein). The use of such reporter elements is well known in the art. Selection of the reporter element to be employed is within ordinary skill in the art, and will depend on factors such as substrate requirements and the desired level of detection sensitivity. Various reporter elements are commercially available, e.g., as a component sequence in a commercial vector. Alternatively, a reporter element can be obtained by screening a DNA library, or by application of conventional PCR techniques.

The reporter element can be inserted into a plasmid, cosmid, vector, yeast artificial chromosome, or other nucleic acid molecule, wherein the reporter element is operably linked to a UPRE. Preferably, the reporter element is under the control of the UPRE only, and not under the control of any other regulatory element that naturally controls expression of the UPR pathway or any other pathway. Variants of reporter elements are also within the scope of the invention, including gene fusion products, truncated genes, and genetically encoded fluorescent tags.

Antibodies

In some embodiments of the invention, antibodies that specifically recognize one or more epitopes of a polypeptide expressed by a UPRE-controlled reporter element can be used to assay for UPRE induction. Such antibodies include polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above.

For the production of antibodies, various host animals may be immunized by injection with the polypeptide encoded by the UPRE-linked reporter sequence, or a polypeptide containing an epitope of the reporter polypeptide. Such host animals may include, but are not limited to, rabbits, mice, and rats, to name but a few. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, (1975, Nature, 256: 495-497; and U.S. Pat. No. 4,376,110), the human B-cell hybridoma technique (Kosbor et al., 1983, Immunology Today, 4:72; Cole et al., 1983, Proc. Natl. Acad. Sci. USA, 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of this invention may be cultivated in vitro or in vivo. Production of high titers of mAbs in vivo makes this the presently preferred method of production.

Alternatively, techniques described for the production of single chain antibodies (U.S. Pat. No. 4,946,778; Bird, 1988, Science, 242:423-426; Huston et al., 1988, Proc. Natl. Acad. Sci. USA, 85:5879-5883; and Ward et al., 1989, Nature, 334:544-546) can be adapted to produce single chain antibodies against β-lactamase. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed (Huse et al., 1989, Science, 246:1275-1281) to allow rapid and easy identification of monoclonal Fab fragments with the desired specificity.

Vector

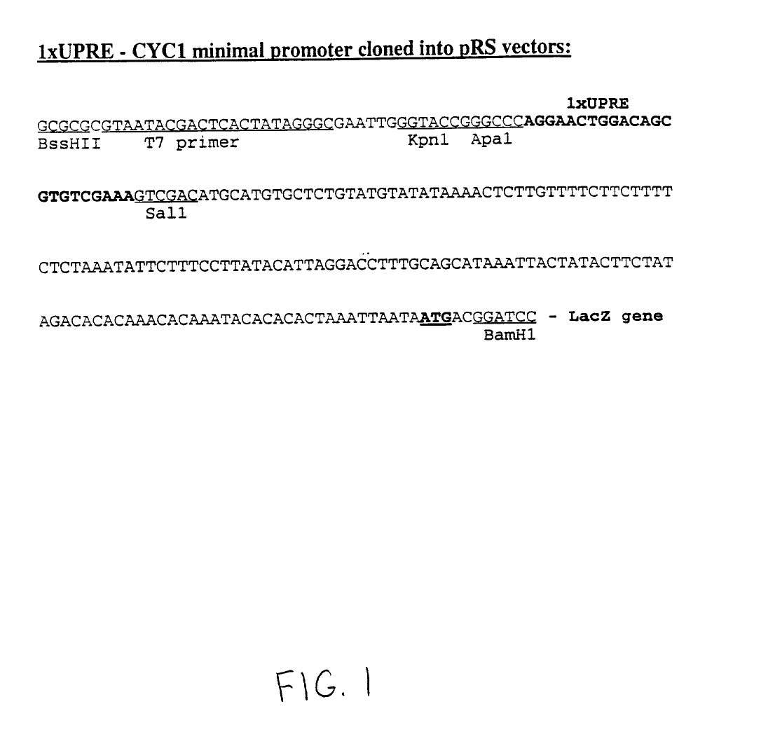

Any suitable yeast expression vector can be employed in practicing the invention. Numerous yeast vectors, including useful commercial vectors, are known in the art. Preferably, the expression vector will include a promoter, e.g., CYCl minimal promoter, a KAR2 promoter (Rose et al., 1989, Cell, 57:1223-1236), or other naturally occurring promoter, appropriately situated with respect to the UPRE. Detailed guidance concerning construction of a vector designated pFY7, which comprises a UPRE operably linked to a lacZ (β-galactosidase) reporter element can be found in PCT publication WO 96/08561. In some embodiments of the invention, the vector contains more than one UPRE operably linked to a promoter and reporter element. A preferred vector contains 3 UPREs (FIG. 1).

Vectors for use in the present invention can be constructed routinely, without undue experimentation, by skilled persons utilizing conventional recombinant DNA technology. For general guidance concerning recombinant DNA technology, see, e.g., Sambrook et al., 1989, Molecular Cloning—A Laboratory Manual (2nd Ed.), Cold Spring Harbor Laboratory Press; Ausubel et al., 1989, Current Protocols in Molecular Biology, Wiley Interscience.

Secondary Screen

The primary (induction) screen, which is based on expression of a reporter element operably linked to a UPRE, detects candidate compounds that inhibit GPI anchoring, but it is not specific for GPI anchoring inhibitors. The primary screen also detects compounds that lead to accumulation of improperly folded proteins by other mechanisms. Therefore, a secondary screen is employed to identify compounds that inhibit GPI anchoring. In this two-step screening process, the primary screen provides a very rapid and convenient mechanism for eliminating a large percentage of screened compounds from further consideration as GPI anchoring inhibitors, without actually subjecting them to a GPI anchoring-specific assay. This is advantageous because the various GPI anchoring-specific assays are often less rapid, economical or convenient than the primary screen.

Any of various assay strategies can be employed in the secondary screen. If biosynthesis of a functional GPI anchor is inhibited, GPI anchoring cannot take place. Therefore, assaying for inhibition of an enzymatic step in GPI anchor biosynthesis provides a convenient and effective approach for detecting certain inhibitors of GPI anchoring. Such assays are known in the art. See, e.g., Leidich et al., 1994, “A conditionally lethal yeast mutant blocked at the first step in glycosyl phosphatidylinositol anchor biosynthesis,” J. Biol. Chem. 269:10193-10196. See also, Takeda et al., 1996, “GPI anchor synthesis,” Trends Biochem. Sci. 20:367-371 (and references cited therein).

Another approach that can be applied in the secondary screen is to assay for the maturation of a major yeast GPI-anchored protein, e.g., Gaslp. Gaslp occurs in a 105 1D form in the endoplasmic reticulum. Upon transport to the Golgi, its core glycan chains are elongated to yield a 125 kD form of Gasip. Maturation of Gaslp can be assayed by extracting total protein from yeast cells, subjecting the protein to SDS-PAGE, transfer of the separated proteins to a nitrocellulose filter decorated with a polyclonal antibody directed against Gaslp. This assay can be performed essentially as described in Suitterlin et al., 1997, EMBO J. 16:6374-6383.

Another approach that can be applied in the secondary screen is to assay for accumulation of a precursor form of Gaslp (or any other GPI-anchored protein). Accumulation of the precursor form can be detected readily by Western blot assay techniques employing an antibody directed against a GPI-anchored protein, e.g., an anti-Gaslp antibody Alternatively, accumulation of the precursor form can be monitored by an immunoprecipitation assay employing such an antibody. See, e.g., Gaynor et al., 1999, Mol. Biol. Cell 10:627-648; Sutterlein et al., 1997, EMBO J. 16:6374-6383.

Another approach that can be applied in the secondary screen is to assay for inositol incorporation into protein by yeast cells. GPI-anchored proteins are the only cellular proteins known to be covalently attached to inositol. Therefore, lack of a signal in such an assay would indicate that the candidate compound is inhibiting GPI-anchor biosynthesis. Assaying for inositol incorporation into protein can be by any suitable means, e.g., by providing radioactively labeled inositol to test cells and detecting protein-bound radioactivity by conventional techniques. See, e.g., Gaynor et al., 1999, Mol. Biol. Cell 10:627-648; Sutterlein et al., 1997, EMBO J. 16:6374-6383. Sutterlein et al., 1998, Biochem J. 332: 153-159.

In addition, the secondary screen can be an assay for accumulation of precursor forms of the GPI anchor. Precursor forms of the GPI anchor can be detected by any suitable means. For example, lipid labeling can be accomplished using [³H]-inositol, [¹⁴C]-mannose, or both, and GPI-anchor precursor forms can be monitored by TLC techniques. See, e.g., Gaynor et al., 1999, supra; Sutterlein et al., 1998, Biochem J. 332:153-159.

Another approach that can be applied in the secondary screen is to assay for overexpression resistance. There are at least 11 known essential genes in the GPI-anchor biosynthesis pathway. Increased resistance due to overexpression of any of these genes could indicate the direct cellular target of a candidate compound. See, e.g., Sutterlein et al., 1998, Biochem J 332:153-159.

The secondary screen can also involve membrane labeling. In an example of this approach, membrane preparations from yeast are labeled with [¹⁴C]-GDP-mannose or [³H]-UDP-GlcNAc and analyzed by TLC to monitor the build-up of precursor intermediates. See, e.g., Sutterlein et al., 1997, EMBO J. 16:6374-6383.

Further information useful in designing and conducting the secondary screen can be found in references including the following: Taron et al., 2000, Mol. Biol. Cell 11:1611 -1630; Benghezal et al., 1996, EMBO J. 15:6575-6583; Leidich et al., 1995, J. Biol. Chem. 270:13029-13035; and Schonbachler et al., 1995, EMBO J. 14:1637-1645.

Data Analysis

Hit thresholds are defined as reporter induction values over a defined amount. For example, a hit rate can be defined as an induction value of 2.0-fold or greater, as compared to the average background, with controls.

Uses of Compounds that Inhibit GPI Anchoring

Compounds discovered using the methods of the invention can be used to treat fungal infections including fungal dermatophytoses and other skin infections associated with the presence of fungi, pulmonary disorders that are caused by hypersensitivity to fungi, fungal infections with pleural involvement, fungal infections involving the genitourinary tract, and systemic fungal diseases (systemic mycoses). Compounds discovered using the methods of the invention also may be useful for treating diseases caused by protozoans, e.g., amebiasis, giardiasis, malaria, leishmaniasis, babeosiosis, and cryptosporidiosis.

To accomplish the foregoing uses, an effective amount of the compound can be administered to the organism. The effective amount of a compound used to practice the present invention varies depending upon the extent, nature (e.g., yeast species, affected organ), and severity of the infection to be treated, the manner of administration, the age, body weight, and other conditions of the organism to be treated, and ultimately will be decided by the attending physician, veterinarian, or experimenter. The effective amount of a compound to be administered can depend on body surface area, weight, and overall condition of the organism. The interrelationship of dosages for animals and humans (based on milligrams per meter squared of body surface) is described by Freireich, et al., 1966, Cancer Chemother. Rep., 50: 219. Body surface area may be approximately determined from patient height and weight. See, e.g., Scientific Tables, Geigy Pharmaceuticals, Ardley, New York, pages 537-538, 1970. An effective amount of the compound for practicing the present invention can range from about 5 μg/kg to about 500 mg/kg, e.g., from about 500 μg/kg to about 250 mg/kg or from about 1 to about 150 mg/kg. Effective doses will also vary, as recognized by those skilled in the art, dependant on route of administration, excipient usage, and the possibility of co-usage with other therapeutic treatments.

The formulations may conveniently be presented in unit dosage form and may be prepared by any of the methods well known in the art of pharmacy. All methods include the step of bringing the active ingredient or ingredients into association with a suitable carrier which constitutes one or more accessory ingredients, unless the compound can be administered in a pure form. In general, the formulations for tablets or powders are prepared by uniformly and intimately blending the active ingredient with finely divided solid carriers, and then, if necessary as in the case of tablets, forming the product into the desired shape and size.

The compounds described here can be administered by any route appropriate to the infection being treated. They can be injected into the bloodstream of the subject being treated, applied topically, or administered orally, subcutaneously, or intraperitoneally. However, it will be readily appreciated by those skilled in the art that the route, such as intravenous, subcutaneous, intramuscular, intraperitoneal, nasal, oral, etc., will vary with the condition being treated and the activity of the compound being used. The invention will be further described in the following examples, which do not limit the scope of the invention described in the claims.

The invention is further illustrated by the following examples. The examples are provided solely for purposes of illustration. They are not to be construed as limiting the scope or content of the invention in any way.

### EXAMPLES

Several UPRE::lacz reporter plasmids were constructed and used to transform a S. cerevisiae Δ mdr mutant and the transformants assessed for their ability to selectively respond to tunicamycin. The reporter sequence 3X UPRE-CYCl_{MIN}lacz (FIG. 2) provided a selective, sensitive, and robust screening response to tunicamycin treatment and was selected for use in the Very High Throughput Screen (vHTS; Example 2). When yeast cells containing this construct were exposed to tunicamycin treatment, the lacZ reporter gene was over transcribed due to the presence of the upstream UPRE sequence. The activity of an associated β-galactosidase reporter gene thus served as an indirect signal of UPR-pathway stimulation.

### Example 1

### Reagents and Solutions

The yeast strains were diploid Δmdr mutants of Saccharomyces cerevisiae: MMB 1663 (3XUPRE CYCl_{MIN}::lacz). Yeast glycerol stocks were prepared by culturing MMB 1663 (3XUPRE CYCl_{MIN}::lacZ) overnight to an OD 600 nm of 0.500. The culture was concentrated by centrifugation at 1000×g for 10 minutes and the pellet resuspended in fresh medium to an OD 600 nm of 1.00. An equal volume of HR Medium with 50% glycerol was added, producing a suspension of cells with an OD 600 nm of 0.500 and a final concentration of 25% glycerol. For freezing, 1.0 ml of the suspension was aliquoted into cryovials and frozen overnight at −20° C., then transferred to −80° C. for long term storage.

Stock solutions of tunicamycin were prepared by diluting a 5.0-mg bottle of tunicamycin (Sigma) with 0.5 ml of dimethyl sulphoxide (DMSO) to a final concentration of 10.0 mg/ml. The stock solution was stored at −20° C.

Lysis Buffer was 0.026% Na deoxycholic acid, 0.053% CTAB, 265 mM NaCl, 395 mM HEPES pH 6.5 and was stored at room temperature.

High Resolution (HR) medium for cell culture was prepared according to the following protocol. Prewarm and dissolve: 0.80 L dH₂O and 0.30 g L-Leucine, (Sigma, L-8000), then add 46.02 g HR Powder (Difco, Custom Order; see Table 1 below), 1.00 g Sodium Bicarbonate (Sigma, S-6014) and adjust the pH to 6.5, then bring the total volume to 2.0 L with dH₂O, and filter sterilize using a 0.2 μM Nalgene PES filter unit. The solution can be stored at 4° C. for up to 2 weeks. Avoid all detergent in glassware and filter units.

TABLE 1
HR Medium

| Component | Grams/Liter |
|---|---|
| Dextrose | 9.990 |
| Potassium phosphate monobasic | 0.995 |
| Ammonium sulfate | 2.495 |
| L-Glutamine | 0.290 |
| Magnesium sulfate, anhydrous | 0.495 |
| Sodium chloride | 0.100 |
| Calcium chloride | 0.100 |
| L-Lysine mono HCl | 0.0365 |
| L-Valine | 0.0235 |
| L-Arginine | 0.0210 |
| DL-methionine | 0.00945 |
| Tryptophane | 0.01000 |
| Inositol | 0.001985 |
| Boric acid | 0.000495 |
| Calcium d-pantothenic acid | 0.000395 |
| Nicotinic acid | 0.000395 |
| Pyridoxine HCl | 0.000395 |
| Thiamine | 0.000395 |
| Manganese sulfate | 0.000395 |
| Zinc sulfate | 0.000700 |
| P-amino benzoic acid | 0.0001975 |
| Riboflavin, USP | 0.0001975 |
| Ferric chloride | 0.0001975 |
| Cupric sulfate | 0.000060 |
| Biotin, crystalline | 0.000002 |
| Folic acid | 0.0001975 |
| L-Isoleucine | 0.02600 |
| Sodium molybdate | 0.000235 |
| Potassium iodide | 0.00010 |
| L-Threonine | 0.02380 |
| MOPS buffer (hemisodium salt) | 15.698 |
| MOPS buffer (free acid) | 15.697 |
| L-Leucine | 0.300 |
| Sodium bicarbonate* | 1.000 |

*indicates reagent added to HR powder to produce final pH 6.5

The tunicamycin (30 μg/ml) Positive Plate Control Solution (3.0% DMSO) was prepared in HR medium. To prepare 10 ml of positive plate control solution, 0.300 ml DMSO and 0.030 ml tunicamycin stock solution (10 mg/ml 100% DMSO) were added to 9.670 ml HR medium. This was prepared fresh daily using the tunicamycin stock solution.

Lysis buffer stock (used for preparing 2X lysis buffer) contained final concentrations of 0.026% Na Deoxycholic acid, 0.053% CTAB (Sigma H5882), 265 mM NaCl, 395 mM HEPES, pH 6.5. The solution was stored at room temperature.

Lysis buffer/β-galactosidase detection buffer was prepared fresh and dispensed within 2 hours of mixing the components. To prepare 100 ml of 2X lysis buffer, the following were mixed: 4.0 ml Galacton-Star (Tropix, Inc. Cat # GS100), 20.0 ml Sapphire II (Tropix, Inc., Cat. # LAX250), and 76.0 ml lysis buffer.

Plates containing the compounds to be tested were flat-bottom 384-well polystyrene plates containing 10 I of 14 mM compounds in 100% DMSO. 30 μl of sterile water was added to these wells to obtain 40 μL of 1 mM samples in 25% DMSO. 5 μl of this sample was then transferred into a well of a clear 384-well polystyrene plate pre-filled with 35 μl of water (1:8 dilution, 125 μM in 3.125% DMSO). The samples were then mixed 4 times and 5 μl of the diluted compound was transferred to Corning flat-bottom white opaque plates. These plates were the Primary Screening Plates. The source and the dilution plates were stored covered at −80° C.

The Primary Screening Plates were dried down overnight to films and stored at −20° C. until used for screening (≦2 months).

### Example 2

### Very High Throughput Screening

Medium Production and Plate Preparation

1. 3.0 L of HR Medium for cell culture (for 250 plates) and 1.0 L of HR Medium was prepared for each 100 plates to be screened. A QC-test was performed overnight for appropriate growth of MMB 1663. 0.050 ml of culture was inoculated into 50 ml of prewarmed HR medium, producing an OD 600 nm of 0.100 to 0.400 after 18-22 hours (h) of growth with shaking at 250 rpm and 30° C. An equal volume of sterile medium was tested under identical conditions. Cultures were monitored for contamination by phase-contrast and dark field microscopy.

2. Primary screening plates were transferred from -20° C. to 4° C.

Medium QC and Control Dispensation

1. A glycerol stock vial of MMB 1663 (3X UPRE CYClₘᵢₙ::lacZ) was removed from a −80° C. freezer and thawed at 30° C. for 5 minutes (min.).

2. Inoculation was performed as follows: 1.0 ml of culture per 1.0 L of prewarmed HR medium, pH 6.5, and incubated at 30° C., with shaking at 250 rpm, for 18-22 hours.

3. 10 ml of 30 μg/ml tunicamycin was prepared and 5 μl of working tunicamycin solution was dispensed to appropriate control wells. The final concentration of tunicamycin was 5 μg/ml and 0.5% DMSO (using 5 μl per control well +25 μl of cell culture).

4. Plates were allowed to warm to room temperature overnight.

Cell Addition and Primary Challenge

1. Overnight cultures were monitored by phase-contrast and dark field microscopy for bacterial contamination, and the procedure was continued if cultures were found to be axenic.

2. HR Medium was prewarmed to 30° C. (1.0 L volumes required about 1 hour to equilibrate).

3. Primary Screening Plates were transferred to 30° C. in 5-plate stacks until all plates reached 30° C. (2 hours).

4. A Multidrop 96/384-well Dispenser (Titertec) was prepared and primed.

5. Overnight culture were diluted to an OD 600 nm of 0.100 with HR Medium (30° C.). Cultures were not used if cell growth was found to exceed 0.600 OD 600 nm (indicating that the cells were in late exponential phase).

6. Using the MultiDrop, 25 μl/well of yeast culture was dispensed into the screening plates and incubated for 4 hours at 30° C. Handlers worked in stacks of 80 plates to maintain a 4-hour time window, avoiding longer incubations (>5 hour), which can result in secondary regulatory reporter inductions.

7. After 4 hours at 30° C., screening plates were transferred to 4° C. Transfers to 4° C. were staggered in a manner similar to the handling of plates in step 6 above.

Lysis/Substrate Addition and β-Galactosidase Luminescence Detection

Working in 80-plate batches, the following guideline was used to assay β-galactosidase activity. The Tropix Northstar Luminescence plate reader processes 384-well plates at a rate of 1.0 min/plate when plates are read for 0.5 minutes per plate. The instrument has an associated 80-plate Twister and can process 80 plates in 80 min. The acceptable window for signal stability is between 90 and 240 minutes (150 minutes total). Other plate readers can be used and the process adjusted appropriately. For example, if using a Packard TopCount, it processes 384-well plates at 2.0 min/plate and batch size for reading plates should be adjusted accordingly.

1. The first 80 screening plates were transferred from the 4° C. cold room to ambient temperature in stacks of 5 plates and allowed 2.0 hours to equilibrate temperature (screening plates were not warmed at temperatures above ambient).

2. During the 2.0 hour period, the plate reader was prepared with fresh Lysis/β-Galactosidase Substrate Mixture, and the Multidrop head was primed with 70% ethanol.

3. After warming the first 80-screening plate batch for 2.0 hours, the second 80-screening plate batch was transferred from 4° C. to ambient temperature in stacks of 5 plates and processed as in batch #1.

4. Using the Multidrop, 25 μl/well of fresh 2X Lysis/β-Galactosidase Substrate mixture was dispensed to the first 80-plate batch. (Packard TopSeal-A Plate Tape is not required for either plate reader when volumes are ≦50 μl for 384-well or ≦200 μl for 96-well plates).

5. After 90 minutes, β-galactosidase activity was read, using a 90 to 240 minute window.

Data analysis is described above wherein a 2-fold or greater level of induction compared to background, e.g., wells processed as above but without a test compound added.

### Example 3

### Use of the UPRE Reporter Screen, Primary Assay

The reporter-based screen was set up to identify compounds that induce the UPR. A total of 855 compounds (small organic molecules) were screened, using a reporter plasmid consisting of the 3X UPRE-CYCl minimal promoter cloned into a pRS416 vector (see FIG. 2) and the very high throughput screening procedure described above. A large number of chemistry attractive hits, i.e. compounds that induced the UPR reporter by at least two-fold, were identified from the primary screen.

The specificity of induction was confirmed by follow-up reporter assays in the screening strain as well as a Δhacl strain. Screening with a Δhacl strain is appropriate because Haclp is the transcription factor that activates the UPR pathway. Thus, in the deletion strain (Δhacl strain), background cells are unable to induce the UPR in response to a stimulating agent.

Compounds were then tested for antifungal (AF) activity (using a standard “halo assay,” wherein each compound is tested for the ability to inhibit the growth of yeast on plated media). Only compounds that were found to have AF activity were subjected to secondary assays to determine the mechanism for the AF activity, to ensure that the compounds act specifically on fungi. Of the 855 compounds screened, 222 (25.9%) were found to induce the UPR reporter plasmid by at least two-fold, and were demonstrated to be antifungal. Based on secondary assay results, a number of compounds were identified that target GPI-anchor biosynthesis.

### Example 4

### Secondary Screening Assays

Inositol-labeling Assays

Inositol labeling ([³H]-inositol in vivo labeling) was used as an initial secondary assay to examine lipid and protein profiles of cells treated with AF compounds identified through the primary screen. The assay permits examination of alteration in inositol containing lipids, and is indicative of whether protein incorporation of inositol has been inhibited. Such inhibition suggests that GPI-anchor biosynthesis is a primary target for the compound, warranting further investigation. Inositol labeling also proved useful in identifying compounds that inhibit general phospholipid (PL) biosynthesis. In the inositol-labeling assay, effective compounds lead to a defect in the uptake of the radiolabel, which is generally manifest as an excess of counts in the growth media. Following purity and chemistry assessment, a total of 105 of the 222 compounds identified as AF through the primary screen were subjected to the inositol-labeling assay. Of the 105 compounds screened, 33 (31.4%) were found to cause defective uptake of the radiolabel.

Mannose-labeling Assays

The mannose-labeling assay ([³H]-mannose in vivo labeling) is a rapid method for examining the 9 steps of GPI-anchor biosynthesis after the addition of the first mannose. A Δpmi40 strain was constructed to allow efficient incorporation of exogenous mannose into growing GPI-anchor precursors.

In vivo mannose labeling proved to be a rapid and robust assay for analyzing lipid profiles. Thus, all compounds that inhibited inositol incorporation into protein were subjected to in vivo mannose-labeling assays. In an exemplary experiment, Δpmi40 cells were treated with 100 μg/ml of the appropriate compound for 30 minutes, after which cells were harvested and the lipids extracted and resolved on TLC plates using Solvent A (10:10:2.5, CHCl₃/MeOH/H₂O).

In addition to GPI-anchor intermediates, Dol-P-Man, MIPC and M(IP)₂C were also labeled in this assay. For example, in DMSO control treated cells, both MIPC and M(IP)₂C species can be observed, as can the complete GPI precursor. When cells were treated with certain members of the compounds isolated via the primary assay (i.e. the various compounds demonstrating antifungal activity), a novel lipid species (“CJ-lipid”) migrated above the MIPC band in extracts prepared from those treated cells. The appearance of the CJ-lipid occurs when cells are blocked at the MCD4 step of GPI-anchor biosynthesis, whether the block is induced genetically, or chemically by the addition of a compound isolated via the primary assay. Mcd4p functions in the addition of ethanolamine onto the first mannose of the GPI-glycan. Data in the literature has shown that the composition of this accumulated lipid is Manₙ-GlcN-(acyl)-PI, where “n” represents two mannose additions. It should be noted that the growing GPI glycan appears to differ in yeast and mammalian cells at this step. In yeast the first ethanolamine is added to a GPI-precursor containing two mannose additions whereas the comparable precursor in mammalian cells contains only one mannose.

Further evidence supporting the published data was obtained using a conditional MCD4 mutant (mcd4-174). In this strain background, the accumulation of the “CJ-lipid” was again detectable upon shift of the cells to the non-permissive temperature. This same lipid species can also be detected in extracts from cells treated with a number of other compounds isolated by the primary assay of the present invention.

A total of 11 compounds that caused defective uptake in the inositol labeling assay were subjected to the mannose-labeling assay. Of the 11 subjected to the assay, 6 (54.5%) compounds gave rise to a novel mannose-labeled lipid intermediate.

Overexpression Resistance Assay

An overexpression resistance (OER) assay was employed to determine the cellular target of compounds isolated via the primary assay. Each of the known genes in the GPI pathway were overexpressed by cloning the gene under the control of its endogenous promoter in 2 micron (multi-copy) vectors. This is a rapid and very powerful assay, but has certain limitations. For example, with a number of hits, precipitation of the compound can be observed when spotted onto plates. Also, while a number of the compounds were AF, they were not extremely potent in yeast and therefore did not give rise to a large zone of inhibition on plates. In such cases, the ability to detect increased resistance in the shoulder region is limited by the small zone size. Further, a relatively high MIC value was observed for many of the compounds. The relatively high MIC value suggests that when this assay is carried out in liquid format there is a minimal window in which to observe increased resistance. Therefore, the OER assay, while very powerful and rapid, may be limiting when working with compounds that are weakly AF.

SDS Hypersensitivity Assay

Published data on a mcd4 mutant strain have shown that it has a number of characteristic phenotypes including increased sensitivity to SDS, an osmotic destabilizing detergent. The presence of as little as 0.004% SDS in the growth media leads to increased sensitivity of a wild type yeast strain to GPI-anchor inhibitors and thereby allows for the rapid screening of analogs. Again, this is a potentially information-rich assay but is subject to the same technical limitations discussed above for the OER assay. In addition, there is a potential for false positives as exemplified by fluconazole, a compound that targets the ergosterol pathway, but gives an unambiguous positive result on SDS plates.

In-vitro GPI Anchor Biosynthesis Assay

Crude lysate from hypotonic lysing of spheroplasts as well as microsomal preparations can be used as an enzyme source for a coupled in vitro assay. Addition of UDP-[³H]-GlcNAc, ATP, Coenzyme A and GDP-mannose provides the necessary substrates to synthesize a complete precursor from endogenous PI and fatty acid. In the in vitro assay, when the components and the inhibitor CJ-19089 are added in a stepwise fashion, the CJ-lipid can be visualized when the products are extracted and resolved using TLC.

Positive Control for Secondary Assays

Studies on a natural compound, CJ-19089 (also known as YW3548, a terpenoid lactone) have documented that this compound targets the MCD4 catalyzed step in the GPI-anchor biosynthesis pathway. CJ-19089 specifically blocks the addition of the third mannose to the intermediate structure Man2-GlcN-acylPI during anchor biosynthesis. Consistent with the block in GPI synthesis, CJ-19089 prevents the incorporation of [³H]myo-inositol into proteins, prevents transport of GPI-anchored proteins to the Golgi apparatus, and is toxic. This inhibitor has proven to be a very valuable tool for examining the pathway and establishing standards for chromatographic analysis. Assays performed (MCD4 overexpression and inositol-labeling experiments in a mcd-4 mutant strain, mcd4-174) support the published literature and indicate that the target of this compound is Mcd4p. CJ-19089 was used as a positive control in each assay and was used in comparison to untreated samples for assay development.

### OTHER EMBODIMENTS

A number of embodiments of the invention have been described. Nevertheless, it will be understood that various modifications may be made without departing from the spirit and scope of the invention. For example, the secondary screen could be based on an assay for attachment of a complete GPI moiety to a protein. Accordingly, other embodiments are within the scope of the following claims.

What is claimed is:

1. A method of identifying a compound that activates the unfolded protein response pathway in fungi, the method comprising:
(a) providing a yeast cell containing a vector comprising at least one unfolded protein response element operably linked to a reporter element;
(b) incubating the yeast cell in the presence of a candidate compound; and
(c) detecting expression of the reporter element in the presence of the candidate compound as compared to expression of the reporter element in the absence of the candidate compound, wherein a 2-fold or greater increase in expression of the reporter element in the presence of the candidate compound indicates that the candidate compound activates the unfolded protein response pathway.

2. The method of claim 1, further comprising subjecting a compound that activates the unfolded protein response pathway to a secondary screen comprising an assay for inhibition of glycosylphosphatidylinositol (GPI) anchoring.

3. The method of claim 1, wherein the yeast cell is a member of the genus Saccharomyces.

4. The method of claim 1, wherein the unfolded protein response element comprises the nucleotide sequence AGGAACTGGACAGCGTGTCGAAA (SEQ ID NO:1).

5. The method of claim 1, wherein the vector comprises one to five unfolded response elements operably linked to a reporter element.

6. The method of claim 5, wherein the vector comprises three unfolded response elements operably linked to a reporter element.

7. The method of claim 1, wherein the reporter element is selected from the group consisting of a β-galactosidase coding sequence and a luciferase coding sequence.

8. The method of claim 2, wherein the secondary screen comprises an enzyme assay for a step in GPI anchor biosynthesis or an assay for maturation of a yeast GPI-anchored protein.

9. The method of claim 2, wherein the secondary screen comprises an assay for detecting inositol incorporation into protein by yeast cells.

10. The method of claim 2, wherein the secondary screen is a lipid labeling assay.

11. The method of claim 10, wherein lipids are labeled with [³H]-inositol, [¹⁴C]-mannose, or both.

12. The method of claim 2, wherein the secondary screen is an overexpression resistance assay.

13. A method of identifying a compound having antifungal activity, the method comprising:
(a) providing a yeast cell containing a vector comprising at least one unfolded protein response element operably linked to a reporter element;
(b) incubating the yeast cell in the presence of a candidate compound;
(c) detecting expression of the reporter element in the presence of the candidate compound as compared to expression of the reporter element in the absence of the candidate compound, wherein a 2-fold or greater increase in expression of the reporter element in the presence of the candidate compound indicates that the candidate compound activates the unfolded protein response pathway; and
(d) assaying a compound that activates the unfolded protein response pathway for inhibition of GPI anchoring, wherein inhibition of GPI anchoring indicates that the compound has antifungal activity.

14. The method of claim 13, further comprising testing the compound for antifungal activity using a halo assay.

15. The method of claim 13, wherein the yeast cell is a member of the genus Saccharomyces.

16. The method of claim 13, wherein the unfolded protein response element comprises the nucleotide sequence AGGAACTGGACAGCGTGTCGAAA (SEQ ID NO:1).

17. The method of claim 13, wherein the vector comprises one to five unfolded response elements operably linked to a reporter element.

18. The method of claim 17, wherein the vector comprises three unfolded response elements operably linked to a reporter element.

19. The method of claim 13, wherein the reporter element is selected from the group consisting of β-galactosidase coding sequence and a luciferase coding sequence.

20. The method of claim 13, wherein the GPI anchoring assay comprises an enzyme assay for a step in GPI anchor biosynthesis or an assay for maturation of a yeast GPI-anchored protein.

21. The method of claim 13, wherein the GPI anchoring assay comprises an assay for detecting inositol incorporation into protein by yeast cells.

22. The method of claim 13, wherein the GPI anchoring assay is a lipid labeling assay.

23. The method of claim 22, wherein lipids are labeled with [³H]-inositol, [¹⁴C]-mannose, or both.

24. The method of claim 13, wherein the GPI anchoring assay is an overexpression resistance assay.

25. A method for treating a fungal infection in an organism, the method comprising administering to the organism a therapeutically effective amount of a compound identified as having antifungal activity by the method of claim 13.

26. The method of claim 25, wherein the fungal infection is selected from the group consisting of fungal dermatophytoses, pulmonary disorders caused by hypersensitivity to fungi, fungal infections with pleural involvement, fungal infections involving the genitourinary tract, and systemic mycoses.

27. A method for treating a protozoan infection in an organism, the method comprising administering to the organism a therapeutically effective amount of a compound identified as inhibiting GPI anchoring by the method of claim 2.

28. The method of claim 27, wherein the protozoan infection is selected from the group consisting of amebiasis, giardiasis, malaria, leishmaniasis, babeosiosis, and cryptosporidiosis.
